(19) United States
(12) Patent Application Publication
(10) Pub. No.: US 2024/0325091 A1
(43) Pub. Date: Oct. 3, 2024

(54) SURGICAL NAVIGATION SYSTEM HAVING IMPROVED INSTRUMENT TRACKING AND NAVIGATION METHOD

(71) Applicant: B. Braun New Ventures GmbH, Freiburg im Breisgau (DE)

(72) Inventors: Jean Stawiaski, Kirchzarten (DE); Amir Sarvestani, Freiburg (DE)

(21) Appl. No.: 18/706,452

(22) PCT Filed: Oct. 28, 2022
(86) PCT No.: PCT/EP2022/080276

§ 371 Date: May 1, 2024

(30) Foreign Application Priority Data
Nov. 2, 2021 (DE) 10 2021 128 478.3

(51) Int. Cl. A61B 34/20; A61B 90/00; A61B 90/20; A61B 90/96

(52) U.S. Cl. CPC A61B 34/20; A61B 90/20; A61B 90/37; A61B 90/96; A61B 2034/2048; A61B 2034/2051; A61B 2034/2055; A61B 2034/2065; A61B 2090/371

(57) ABSTRACT

A navigation system can be used during surgical intervention on a patient to track a medical instrument. The system includes a display, a medical instrument, a data provision unit for providing 3D digital recording data, an imaging device having a recording head for producing a recording of a surgical area and for detecting and tracking the instrument, a tracking system for detecting and tracking the recording head and detecting a portion of the patient for registration with the 3D recording data, and a control unit for processing data from the imaging device and tracking system, and the 3D recording data, determining a position and/or orientation of the instrument, producing a correlation display using the 3D recording data and the position and/or orientation of the instrument, and outputting it through the display. A navigation tower, method and computer-readable storage medium can include or be used with the navigation system.

### CROSS-REFERENCE TO RELATED APPLICATIONS

This application is the United States national stage entry of International Application No. PCT/EP2022/080276, filed on Oct. 28, 2022, and claims priority to German Application No. 10 2021 128 478.3, filed on Nov. 2, 2021. The contents of International Application No. PCT/EP2022/080276 and German Application No. 10 2021 128 478.3 are incorporated by reference herein in their entireties.

### FIELD

The present disclosure relates to a surgical navigation system for navigation during a surgical intervention on a patient for tracking at least one medical instrument used during the intervention. In addition, the present disclosure relates to a navigation tower, a navigation method and a computer-readable storage medium.

### BACKGROUND

Surgical navigation can usually only be performed with special instruments that have special marking systems with markers, such as infrared reference points, or an electromagnetic tracking system. These instruments are known as indicators or pointers and are specially adapted to mark a position in three-dimensional space with their tip, which is detected by the navigation system.

In order to obtain the required navigation information during an intervention, a user, such as a surgeon, has to interrupt his process sequence, take the specially adapted (pointer) instrument in his hand and guide it to the desired location, wherein the only function of the (pointer) instrument is to provide navigation information. Such a continuous change between the individual instruments prolongs an intervention on a patient in a disadvantageous way and also leads to fatigue on the part of the surgeon. In addition, a large number of medical instruments with different functions always have to be available.

In addition, there is currently the problem that navigation systems usually use an external camera system, such as a stereo camera, which is positioned at a distance from an intervention region. In a surgical navigation system, for example an infrared-based navigation system, a field of view within lines of sight is therefore severely limited in a complex environment of an intervention region, in particular when a navigation system and a surgical microscope are used simultaneously. The tracing of the instrument is temporarily interrupted and the precision of the tracing also decreases. In addition, restrictions regarding the field of view to be kept free for the camera system need to be observed, which further complicates the intervention on the patient.

### SUMMARY

Therefore the objects and objectives of the present disclosure are to provide a surgical navigation system, a navigation tower, a navigation method as well as a computer-readable storage medium that avoids or at least reduces the disadvantages of the prior art. In particular, a surgical navigation system as well as a navigation method are to be provided that provide a higher precision of tracking of a medical instrument and allow continuous and uninterrupted tracing as well as an even better detection of an intervention region. A user, in particular a surgeon, is to receive a better and safer navigation modality. In addition, a partial object is to support a surgical process sequence even better and to perform it faster. Another partial object can be seen in using existing surgical instruments as pointers or at least only having to change them slightly, in particular only supplementing them. Another partial object is to increase the available navigation time during an intervention.

The objects of the present disclosure are solved with respect to a generic surgical navigation system according to the invention, with respect to a generic navigation tower according to the invention, with respect to a navigation method according to the invention, and with respect to a computer-readable storage medium according to the invention.

A basic idea of the present disclosure can thus be seen in that the surgical navigation system and navigation method do not detect a medical instrument directly and absolutely via, for example, a camera system, but indirectly via, on the one hand, the tracing of the imaging head of the medical imaging device, which is arranged closer to the intervention region than, for example, the camera system, and, on the other hand, the tracing of the medical instrument by the imaging head itself, so that the position and/or orientation of the instrument is computable or determinable via a serial linking of the two trackings. If the patient is registered to the navigation system, 3D imaging data/3D image data, in particular preoperative 3D imaging data such as MRI images and/or CT images, are also registered to the patient and the determined position and/or orientation of the instrument can be determined in the 3D imaging data for navigation and can then displayed to the user.

In a sense, decoupling of the direct tracing of the instrument with at least two-part, sequential tracing is provided, i.e. the tracing of the imaging head and an independent tracing of the instrument by the imaging head. The two tracings provide two individual (coordinate) transformations that are linked together in order to obtain a transformation for the instrument to be tracked. Due to the independence, different tracing systems or methods may also be used, so that, for tracing the imaging head, a tracking system configured for it and adapted for greater distances may be used, while different and specially adapted tracing for a shorter distance in the area of the intervention may also be used for tracing the instrument. In this way, even common, e.g. standardized medical, in particular surgical, instruments can be detected, which is not possible in the prior art.

This at least coupled/linked two-part or two-stage, serial tracing can achieve a particularly high precision of tracking/a high tracing accuracy due to the small distance between (the imaging head) of the medical imaging device, in particular a stereomicroscope, and the instrument used. In this way, the problems with a line of sight with an external navigation camera, for example, are also avoided, since the navigation camera does not need to see the instrument to be tracked itself, but only the imaging head of the medical imaging device, in particular a microscope head with an optical system of a surgical microscope. It is also advantageous that the surgical process sequence is not interrupted by the use of the navigation system, since the detection and tracing of the instrument by the imaging head of the imaging device means that standard instruments can also be followed and do not have to be replaced by navigated instruments such as a pointer.

An intervention with navigation method is furthermore even safer, since the net navigation time available to the surgeon is further increased. Standard surgical instruments are navigated throughout their entire use.

In other words, a surgical navigation system for navigation in a surgical intervention on a patient for tracing of at least one object, in particular a medical instrument, is provided, comprising: a display device, in particular a monitor, for displaying a visual content; at least one object to be tracked, in particular a medical instrument; a medical imaging device having an imaging head, which is adapted to create an optical/visual image of an intervention region in a patient as well as to detect and to trace/to track the object to be tracked, in particular a medical instrument, in the surgical intervention region of the patient with respect to the imaging head; an (external) tracking system, which is adapted to detect at least the imaging head of the imaging device and to track it with respect to the tracking system, as well as to detect and in particular to track at least a partial portion of the patient with the intervention region for registration; a data provision unit, in particular a storage unit, which is adapted to provide digital 3D imaging data, in particular preoperative 3D imaging data, of the patient; and a control unit, which is adapted to process the data of the imaging device, the data of the tracking system as well as the provided 3D imaging data and to determine a position and/or orientation of the object to be tracked, in particular an instrument, in particular an instrument tip of the instrument, by linking (a transformation or transformation matrix of) the tracing from the tracking system to the imaging head as well as the tracing from the imaging head to the object, in particular instrument, and to transfer this to the 3D imaging data of the patient registered to the tracking system and to output this visually by the display device. The control unit can create a correlation representation with the 3D imaging data registered for the patient and this determined/calculated position and/or orientation of the instrument and can output this visually by the display device. In this way, the surgeon can display the (virtual) instrument, in particular the exact position of the instrument tip, in the 3D imaging data on an OR monitor, for example.

Based on the detected patient, the detected imaging head and the instrument detected by the imaging head, a position and/or orientation of the instrument, in particular of an instrument tip, in relation to the 3D imaging data can be determined. In this way, it is possible to determine the position and/or orientation of surgical instruments relative to 3D imaging data (3D image) of the patient.

Thus, a navigation system is provided for following surgical instruments and indicating their position, in particular their pose, relative to 3D imaging data (3D image set) of the patient during surgery, wherein the instrument is followed by an optical system, the optical system is in turn followed by a tracking system of the navigation system, and the patient is also followed by the navigation system (for registration to the 3D imaging data).

The term ‘position’ means a geometric position in three-dimensional space, which is specified in particular via coordinates of a Cartesian coordinate system. In particular, the position can be specified by the three coordinates X, Y and Z.

The term ‘orientation’ in turn indicates an alignment (such as at the position) in space. It can also be said that the orientation specifies an alignment with an indication of a direction or rotation in three-dimensional space. In particular, the orientation can be specified using three angles.

The term ‘pose’ includes both a position and an orientation. In particular, the pose can be specified using six coordinates, three position coordinates X, Y and Z and three angular coordinates for the orientation.

The term 3D defines that the image data is spatial, i.e. three-dimensional. The patient's body or at least a partial region of the body with spatial extension can be digitally available as image data in a three-dimensional space with a Cartesian coordinate system (X, Y, Z).

The medical instrument to be tracked may be a suction tube or forceps, for example, whose distal instrument tip can be used to define a point in space with particular precision. The surgical navigation system may of course also be used to track (smaller) medical devices.

Advantageous embodiments are explained in particular below.

According to a preferred embodiment, the medical imaging device may be a surgical microscope for surgery and/or a medical endoscope/surgical endoscope adapted to perform spatial detection for tracing, and in particular comprising a 3D-camera system for detecting depth information. In craniotomy, for example, a surgical microscope supports the surgeon during the intervention. The position of the microscope head of the surgical microscope is followed with the tracking system of the surgical navigation system. In particular, a surgical stereo microscope is used during the intervention in order to recognize surgical instruments and to calculate their relative position with respect to the microscope's image sensors. The imaging device (vision system) may thus be a surgical microscope or a surgical endoscope. In particular, the surgical navigation system may therefore be used in conjunction with a surgical microscope, in particular a stereo microscope, wherein the control unit is specially adapted to provide corresponding navigation data. With a (conventional) surgical microscope, a part of the intervention region of interest can be targeted down to a distance of a few centimeters and the instrument can be localized and tracked with particular precision.

According to a further preferred embodiment, the imaging head of the medical imaging device may comprise a stereo camera for a stereo image and, in particular, the control unit may be adapted to detect a spatial, three-dimensional position and/or orientation of the instrument, in particular of the instrument tip, relative to the imaging head from the stereo image via machine vision. In other words, the imaging device may have a stereo camera system and/or a 3D camera system with depth information. This means that only the control unit for the evaluation needs to be adapted accordingly and standardized devices such as stereo microscopes or endoscopes with a stereo camera on the end face can be used.

According to a further configuration example of the disclosure, the navigation system may comprise an image analysis device, which is adapted to perform a spatial three-dimensional detection of an instrument from at least two image perspectives, in particular a stereo image, via machine vision. In other words, the navigation system may have a camera system with at least one camera and perform three-dimensional detection and tracing of the instrument on the basis of machine vision.

Preferably, the control unit may be adapted to determine a three-dimensional position and/or orientation of the instrument via image processing (analysis) techniques and/or via triangulation of the stereo image/a stereo picture and/or via reconstruction of a disparity overlap of a stereo image. In particular, the control unit may also be adapted to perform navigation using principles of 3D reconstruction and position determination from stereo images. In particular, for each pixel in the left image of the stereo image, a corresponding pixel is searched for in the right image. The positions of these two pixels are used to calculate the apparent depth of the pixel. Alternatively or additionally, the pose (3D position) of a surgical instrument in relation to the imaging head can be calculated using image processing techniques. In particular, the pose (3D position) of the instrument can be determined by recognizing the instrument in the left and right image according to the principle of triangulation. Alternatively or additionally, the position and/or orientation can be determined by reconstructing a disparity overlap of the stereo images. In such a depth map/disparity map, an instrument is recognized and its pose (3D position) is calculated directly from the corresponding depth values. The recognition of instruments may be performed using the following methods: image processing methods for each individual color image of the left image or of the right image; image processing methods that use a pair of the left image and of the right image (simultaneously); image processing methods that use a single disparity map/depth map; or image processing methods that use a combination of the aforementioned image processing methods. In particular, methods of machine vision include deep learning methods using neural networks or image transformations (vision transformers); hand-crafted design features such as line detection or color detection; or an adaptation of 3D models to images. In particular, the at least one instrument is tracked, preferably by machine vision, by a surgical (surgical stereo) microscope and the surgical microscope itself is tracked by a tracking system of the navigation system.

In particular, a pre-determined/defined optical pattern, which is detected by the imaging head, may be provided, in particular integrated, on an optically visible outer side of the medical instrument to be tracked, in particular on a distal end or end portion of the instrument. The control unit is in turn adapted to recognize and decode the optical pattern or to compare it with a reference stored in a storage unit and to determine a position of an instrument tip relative to the optical pattern or a geometry of the instrument on the basis of the detected optical pattern. The control unit can therefore use the optical pattern to obtain information on a position of the instrument tip or on a geometry of the instrument. The at least one instrument therefore has a predefined optical pattern/optical marking, which is integrated in particular in the distal end or end portion and is detected by the imaging head. The information conveyed via the optical pattern can be useful, since the recognition of the exact position of the instrument tip is difficult due to covering of the instrument tip or a low contrast of the instrument. The recognition of a determined optical pattern on a main body portion of the instrument, on the other hand, is much easier and the control unit can use the information to deduce the position of the instrument tip. One advantage is that even better navigation ergonomics are provided. In particular, the surgical instruments used in the navigation system are not or only slightly modified, in particular by a marking, preferably on or in the area of the tip. This relatively simple and small modification is carried out in order to follow the instrument even more precisely compared to classic navigated instruments with large rigid bodies. For example, direct optical marking can be achieved by simply engraving specific patterns on the instrument, such as QR codes, data matrix codes, barcodes, lines, dots or textures. The optical markings may be QR codes, at least two rings with a predefined distance between them or another unique pattern that the control unit can decode and, in particular, link to stored information. In particular, these optical markings may be used to encode the position of the instrument tip and/or to indicate the geometry of the instrument. In particular, the pre-determined optical pattern may be a QR code and a distance from the QR code to the instrument tip may be encoded in the QR code so that the position of the instrument tip is determinable.

According to a further embodiment, the optical pattern may be a QR code, wherein a distance from the QR code to the instrument tip is encoded in the QR code, so that the position of the instrument syringe is determinable.

In particular, image processing techniques may be used to learn the visual appearance of common surgical instruments in order to deliver optimal recognition accuracy.

According to one embodiment, a geometric shape of the at least one medical instrument, in particular of several medical instruments, may be stored in a storage unit, in particular by an initial three-dimensional detection by the imaging head and/or by the tracking system, and the control unit may determine the position, in particular pose, of the distal tip on the basis of a partial portion of the instrument to be tracked detected by the imaging head and the stored geometric structure. In particular, the recognition of instruments may be simplified by standardizing the visual or geometric appearance of the at least one instrument. In this way, the instruments do not have to be changed and the known, stored geometric shape information is used to recognize the 3D shape of the instrument and finally to determine the pose of the instrument.

Preferably, the tracking system may comprise an infrared-based camera system and/or electromagnetic-based system and/or an IMU (Inertial Measurement Unit)-based tracking system. In the case of an IMU (Inertial Measurement Unit), this may be arranged in particular in the imaging head in order to track the imaging head.

The objects of the present disclosure are solved with respect to a mobile medical navigation tower in that it comprises: a navigation system according to the present disclosure; and a mobile base/mobile cart with wheels for mobile placement of the navigation tower. Due to the design as a compact mobile unit, the navigation tower can be placed flexibly at different locations in an operating room. Thus, a mobile medical and wheeled cart with the navigation system according to the present disclosure is proposed, which in particular comprises a computer, which implements the control unit, and a monitor.

With regard to a navigation method for navigation in a surgical intervention for a patient for tracing/tracking of at least one medical instrument, in particular in a surgical navigation system of the present disclosure, the objects are solved by the steps:
registering a partial portion of a patient, in particular of the patient, with respect to 3D imaging data of the patient;
detecting and tracing the instrument to be tracked by an imaging head of a medical imaging device;
detecting and tracing the imaging head of the medical imaging device by a tracking system;
determining/calculating a position and/or orientation of the medical instrument by linking the tracing of the imaging head and the tracing of the medical instrument;
in particular, transferring the detected position and/or orientation of the medical instrument to the 3D imaging data (3DA); and
outputting a combination display of the 3D imaging data with at least the position and/or orientation of the medical instrument by a display device.

According to an embodiment, the navigation method may further comprise the steps of: creating a stereo image by the medical imaging device; creating, based on the stereo image, a depth map with depth information by an image processing technique and/or by a triangulation and/or by a reconstruction of a disparity overlap; and determining the position and/or orientation of the instrument based on the stereo image and the depth map.

In particular, if the pre-determined optical pattern is a QR code and a distance from the QR code to the instrument tip is encoded in the QR code, the navigation method may further comprise the steps of: decoding the QR code; reading the distance to the instrument tip; determining the position of the instrument tip relative to the imaging head and via the tracked imaging head relative to the 3D imaging data.

With respect to a computer-readable storage medium, the objects are fulfilled by comprising instructions which, when executed by a computer, cause the computer to perform the method steps of the navigation method according to the present embodiment.

Any disclosure related to the navigation system of the present disclosure also applies to the navigation method of the present disclosure and vice versa.

### BRIEF DESCRIPTION OF THE DRAWINGS

The present invention is explained in more detail below with reference to the accompanying Figures via preferred configuration examples.

FIG. 1 shows a schematic view of a surgical navigation system of a preferred embodiment with a surgical stereo microscope;

FIG. 2 shows a detailed schematic partial view of the navigation system from FIG. 1 with tracing of the instrument by an imaging head of the surgical microscope;

FIG. 3 shows a schematic view of the stereo image of the microscope head of FIGS. 1 and 2, in which a surgical instrument is detected;

FIG. 4 shows a schematic view of a 3D reconstruction by triangulation of the stereo image from FIGS. 1 to 3;

FIG. 5 shows a schematic view of a determined depth map of the stereo image for a three-dimensional detection of the instrument to be tracked;

FIG. 6 shows a surgical navigation system of a further, second preferred embodiment, in which a QR code is visibly attached to a distal end portion of the instrument;

FIG. 7 shows a surgical navigation system of a further, third preferred embodiment, in which spaced ring groups are attached to a distal end portion of the instrument and are visible;

FIG. 8 shows a mobile navigation tower with a surgical navigation system of a further, fourth preferred embodiment; and

FIG. 9 shows a flow diagram of a navigation method of a preferred embodiment.

The Figures are schematic in nature and are only intended to aid understanding of the invention. Identical elements are marked with the same reference signs. The features of the various embodiments can be interchanged.

### DETAILED DESCRIPTION

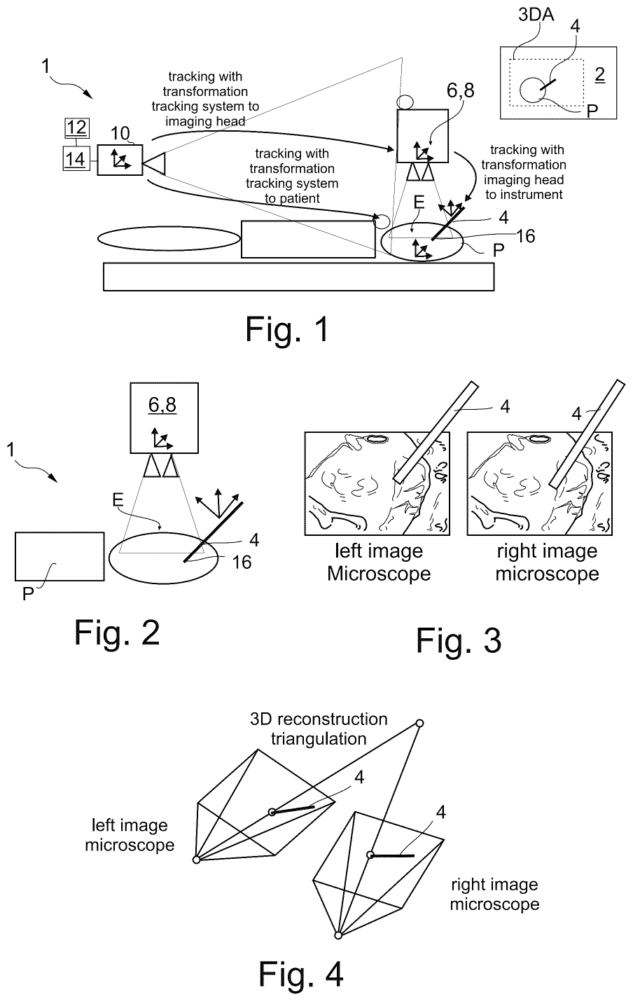

FIG. 1 shows a schematic side view of a surgical navigation system 1 for navigation during a surgical intervention on a patient P. The navigation system 1 has a display device 2 in the form of a surgical monitor for visual outputting of the navigation information to the surgeon.

In an intervention region of the patient, here on the patient's head, the surgeon acts with a medical instrument 4, for example a pair of forceps, a suction tube or a sealing instrument, which he wishes to track with the navigation system 1, and performs the intervention accordingly.

A surgical stereo microscope (hereinafter referred to only as microscope) 6 as a medical imaging device has a (stereo) microscope head as imaging head 8, which has an optical system (not shown) with downstream sensors such as CMOS sensors or CCD sensors in order to create a visual stereo image A (two video recordings at a distance from each other from different positions) of a part or portion of the intervention region E in the patient P. In addition to the intervention region itself, the microscope 6 also detects the medical instrument 4 to be tracked, which is used in the intervention region E by the surgeon. As will be explained in more detail later, the navigation system 1 can use the stereo image A to spatially detect and trace or respectively track the position and orientation of the instrument, i.e. its pose in relation to the imaging head 8.

An external tracking system 10 of the navigation system 1, in particular an infrared-based tracking system 10, in the form of an external (stereo) camera, which is arranged in the area of the patient's legs, in turn detects the imaging head 8 of the microscope 6 and can detect and track it in three dimensions, in particular if infrared markers are attached to it.

A data provision unit in the form of a storage unit 12, such as an SSD memory, provides preoperative, digital 3D imaging data 3DA in the form of MRI images and/or CT images of the patient P for navigation. Patient P is detected by the tracking system 10, for example infrared markers are placed on the patient's head, and registered with respect to the 3D imaging data 3DA. In this way, the ‘real’ current time data, in particular the stereo image A, is correlated with the ‘virtual’ 3D imaging data 3DA. The tracking system 10 also detects a possible movement of the patient's body during the intervention and registers the patient's movement again on the 3D imaging data 3DA.

In the present case, a control unit 14 of the navigation system 1 is specially adapted to process the data of the imaging device 6, the data of the tracking system 10 and the 3D imaging data 3DA provided and, by linking the tracing of the tracking system 10 to the imaging head 8 and the tracing of the imaging head 8 to the instrument 4, to determine a pose of the instrument 4 to be tracked and also a position of an instrument tip 16 of the instrument 4. Specifically, the tracing of the imaging head 8 (microscope head) by the tracking system 10 provides a first transformation matrix in order to infer the current local coordinate system of the imaging head 8 from the local coordinate system of the tracking system 10, in this case a local coordinate system of the external (stereo) camera. The tracing (tracking) of the instrument 4 by the imaging head 8 in turn provides a second transformation matrix from the local coordinate system of the imaging head 8 to the local coordinate system of the instrument 4. The control unit 14 processes this first and second transformation matrix into a total transformation matrix from the local coordinate system of the external (stereo) camera to the instrument 4 itself.

In addition, the patient P was registered by the tracking system 10, i.e. a relation of a local coordinate system of the patient P to the local coordinate system of the external (stereo) camera was detected, which is provided to the control unit 14 as a patient-side transformation matrix. In addition, the patient P is registered to the 3D imaging data 3DA, so that (ideally) the real patient P corresponds to the 3D imaging data 3DA.

The total transformation matrix from the instrument 4 (via imaging head 8) to the external camera, and further from the external camera to the patient P or to the 3D imaging data 3DA, allows the pose (position and orientation) of the instrument 4 to be tracked to be transferred to the 3D imaging data 3DA and displayed accordingly for navigation. The control unit creates a correlation representation which, on the one hand, has the 3D imaging data 3DA registered for the patient P and, on the other hand, also displays the position and/or orientation of the instrument 4, in particular a virtual geometric model of the instrument 4, in the 3D imaging data 3DA (position-correct or orientation-correct, in particular pose-correct). This correlation representation is then output by the OR monitor and the surgeon can see at any time where the instrument 4 with its instrument tip 16 is located in the patient P, even if the instrument tip 16 is not visible.

By combining two tracings, the surgeon can be provided with particularly flexible and safe navigation. Usually, a field of view of the microscope 6 is perpendicular to an intervention region E with the tissue, so that there is virtually no occlusion by other objects. Standardized instruments 4 to be tracked may also be used for navigation during the intervention, since no special pointer instruments are required due to the good visual detection by the microscope 6 with the associated possibility of tracing.

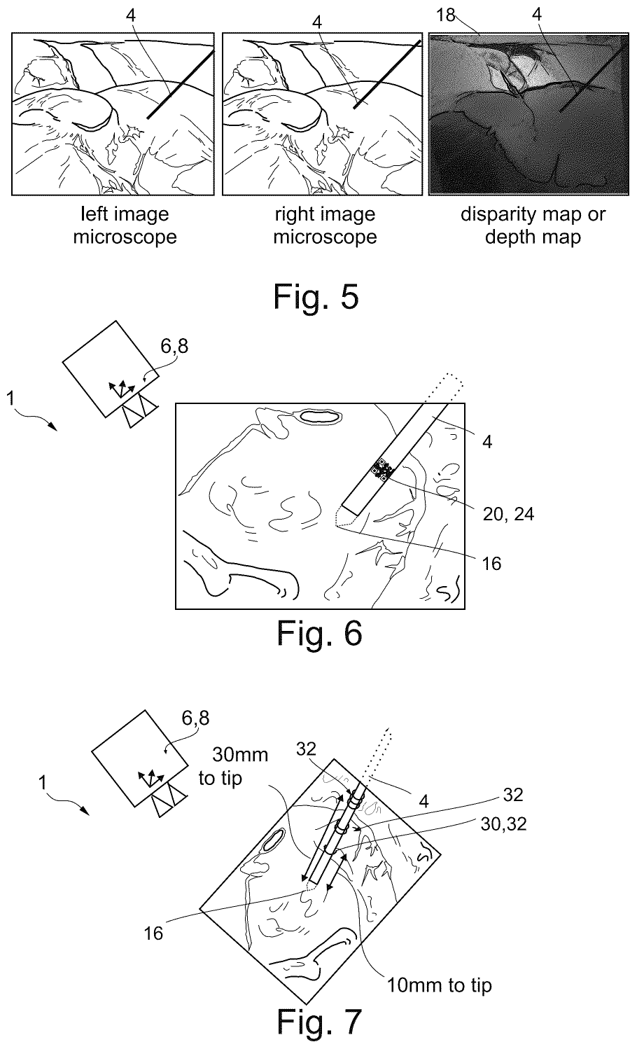

In order to explain the tracing of the instrument 4 by the imaging head 8, FIGS. 2 and 3 show the microscope 6 of FIG. 1 in a detailed partial view as well as an exemplary (two-dimensional) stereo image A. The imaging head 8 of the microscope 6 has an optical system and two downstream, spaced-apart image sensors, which subsequently provide two (slightly) different images (left and right) from the correspondingly different perspectives, as shown schematically in FIG. 3. Using image analysis of the left and right images, a depth can then be determined, as explained below with reference to FIGS. 4 and 5.

FIG. 4 schematically explains the principle of a 3D reconstruction from the two two-dimensional images (stereo image A with left and right image). The principle of triangulation is used to determine depth information for a three-dimensional reconstruction. In this way, the stereo image can be used for spatial, three-dimensional detection, in particular the three-dimensional detection of the instrument 4 to be tracked. The tracing of the instrument 4 is carried out by the control unit 14. In this embodiment, it is therefore sufficient if the surgical microscope 6 merely provides the stereo image A to the control unit 14, so that the control unit 14 determines the pose of the instrument 4 relative to the imaging head 8, or more precisely to the sensors, based on this using the image analysis described above.

Thus, by linking the tracing of the instrument 4 by the imaging head 8 on the basis of an image analysis and the tracing of the imaging head 8 by the tracking system 10 and the 3D imaging data 3DA registered for the patient P, the pose of the instrument 4, in particular the position of the instrument tip 16, can be displayed in the 3D imaging data in a particularly simple and reliable manner.

FIG. 5 again shows a 3D reconstruction based on the stereo image A. For each pixel in the left image, a corresponding pixel is searched for in the right image (or vice versa), whereby a depth of the pixel is calculated by the control unit 14 on the basis of these two pixels. If this is carried out for each pixel of the stereo image A, a depth map 18 (schematically indicated in the right-hand image in FIG. 5) is finally produced, which, together with the left-hand and right-hand images of the stereo image A, can be used to determine a spatial three-dimensional structure and thus also to detect the pose of the instrument 4 and the position of the instrument tip 16.

FIG. 6 schematically shows a surgical navigation system 1 according to a further, second preferred embodiment with an exemplary image. The navigation system 1 differs from that shown in FIGS. 1 to 5 only in that it has a specially adapted instrument 4 to be tracked with an optical marking 20 and the control unit 14 is adapted to assign information to the optical marking and to use it to determine a position of an instrument tip 16.

Specifically, a QR code 24 is engraved in a distal end region 22 of the instrument 4 on a lateral surface/side surface 26 of the instrument 4. This allows a standardized instrument 4 to be subsequently modified and adapted, for example, whereby only an engraving that is particularly simple and quick to produce, for example via a laser, needs to be made. The QR code 24 contains encoded information that the control unit can decode and interpret. On the one hand, a distance in cm can be encoded directly in the QR code 24, so that the position of the instrument tip 16 can be inferred directly from the position of the QR code 24 along a longitudinal axis 28 of the instrument 4 with the amount of the distance, independently of an evaluation program, or the QR code can have an ID as a reference in order to infer the position of the instrument tip 16 via data stored in the storage unit 12, which also have a distance from the QR code 24 to the instrument tip 16. In this way, the instrument tip 16 can also be displayed in the 3D imaging data 3DA without direct visual contact in order to support the surgeon in navigation.

FIG. 7 shows another preferred embodiment of a surgical navigation system. In contrast to the second embodiment with the QR code 24, the instrument 4 to be tracked has circumferential rings 30, which are combined in ring groups 32. The ring groups 32 are arranged along the longitudinal axis 28 of the instrument 4 with circumferential rings 30 and encode a distance from the corresponding ring group 32 to the instrument tip 16. A first ring group 32 with a single circumferential ring is arranged at a distance of 10 cm, a second ring group 32 with two rings at a distance of 20 cm, and a third ring group 32 with three rings 30 at a distance of 30 cm. The control unit 14, on the other hand, is adapted to determine the distance to the instrument tip 16 on the basis of the ring groups 32.

Via the optical markings 20 on portions of the instrument 4 that are particularly easy to see, a position, in particular a pose, of the associated instrument tip 16 can be encoded with respect to the optical marking and possibly also with respect to characteristic design features or even geometry information of the instrument 4 can be encoded directly or indirectly via a database.

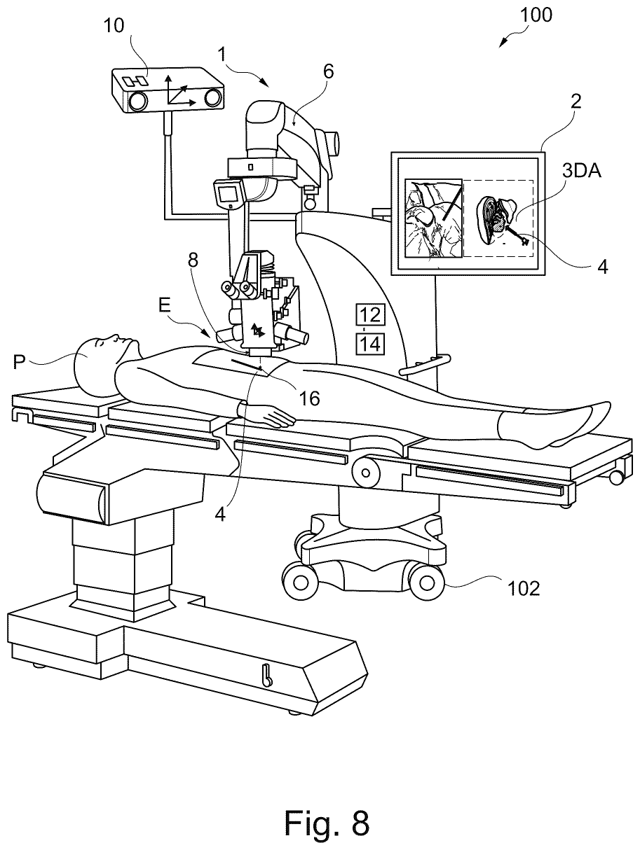

FIG. 8 shows a mobile navigation tower 100 with a surgical navigation system 1 of a further, fourth preferred embodiment. The configuration with wheels 102 allows the navigation tower 100 to be used flexibly at different locations in an operating theater. On the monitor, the surgeon can display a side-by-side representation of the image of the microscope and the correlation representation with the 3D imaging data 3DA and the superimposed pose of the instrument 4.

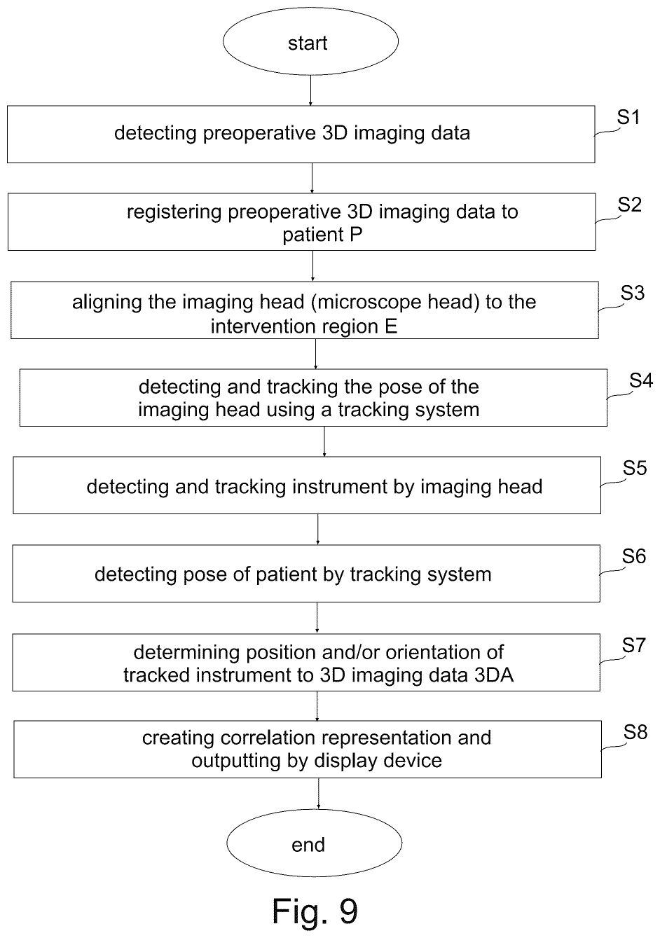

FIG. 9 shows in a flowchart, a navigation method according to a preferred embodiment, which can be performed in a surgical navigation system, in particular in a navigation system 1 described above.

In a first step S1, preoperative 3D imaging data of the patient P is detected.

In a second step S2, the patient P is registered to the 3D imaging data 3DA.

Preferably, in a step S3, the imaging device 6 (visualization system) or the imaging head 8 is oriented to the intervention region E on the patient P.

In a step S4, the imaging head of the imaging device is localized and tracked by the tracking system.

In a step S5, a (three-dimensional) pose (position and orientation) of the instrument relative to the imaging head 8 is determined via the imaging device, in particular via the stereo image.

Preferably, the surgical instrument 4 can be guided by the surgeon in the intervention region E in one step.

Preferably, in a step S6, the patient is detected and localized three-dimensionally by the tracking system 10.

In a subsequent step S7, the position and/or orientation of the instrument 4 relative to the patient P and thus to the previously registered 3D imaging data 3DA is calculated.

Finally, in a step S8, a correlation representation is generated and the position and/or orientation of the instrument 4 is displayed in the 3D imaging data 3DA and output via the monitor.

### LIST OF REFERENCE SIGNS

1 surgical navigation system
2 display device
4 medical instrument
6 imaging device/stereo microscope
8 imaging head
10 tracking system
12 storage unit
14 control unit
16 instrument tip
18 depth map
20 optical marking
22 distal end region of the instrument
24 QR code
26 side surface
28 longitudinal axis of the instrument
30 ring
32 ring groups
100 navigation tower
102 wheels
P patient
E intervention region
A (stereo) image
3DA 3D imaging data
S1 step detecting preoperative 3D imaging data
S2 step registering patient to 3D imaging data
S3 step orienting imaging head on intervention site
S4 step tracing of imaging head by tracking system
S5 step tracing of instrument by imaging head
S6 step detecting pose patient by tracking system
S7 step determining position and/or orientation Instrument to 3D imaging data
S8 step creating correlation representation and outputting by display device

1.-14. (canceled)

15. A surgical navigation system for navigation during a surgical intervention on a patient for tracking of at least one medical instrument, comprising:
a display device, in particular a monitor, for displaying visual content;
at least one medical instrument to be tracked;
a data provision unit, in particular a storage unit, which is adapted to provide digital 3D imaging data, in particular preoperative 3D imaging data, of the patient;
a medical imaging device having an imaging head;
a tracking system, which is adapted to detect and track the imaging head of the imaging device as well as to detect and in particular track at least a partial portion of the patient for registration to the 3D imaging data, wherein:
the imaging head of the medical imaging device is adapted to create an image of a portion of an intervention region of the patient as well as to detect and to track the medical instrument to be tracked with respect to the imaging head; and
a control unit, which is adapted to process the data of the imaging device, the data of the tracking system as well as the provided 3D imaging data and to determine a position and/or orientation of the instrument to be tracked, in particular an instrument tip of the instrument, by linking the tracking from the tracking system to the imaging head as well as the tracking from the imaging head to the instrument and to create a correlation representation with the 3D imaging data registered for the patient and the position and/or orientation of the instrument, in particular the instrument tip, and to output this by the display device.

16. The surgical navigation system according to claim 15, wherein the medical imaging device is a surgical microscope for surgery or a medical endoscope adapted to perform three-dimensional detection for tracking, in particular comprising a 3D-camera system for detecting depth information.

17. The surgical navigation system according to claim 15, wherein the imaging head of the medical imaging device comprises a stereo camera for a stereo image and, in particular, the control unit is adapted to detect a position and/or orientation of the instrument, in particular the instrument tip, relative to the imaging head from the stereo image via machine vision.

18. The surgical navigation system according to claim 17, wherein the control unit is adapted to determine the position and/or orientation of the instrument via triangulation of the stereo image and/or via reconstruction of a disparity overlap of the stereo image.

19. The surgical navigation system according to claim 15, wherein a pre-determined optical pattern, in particular a QR code and/or a barcode and/or two rings spaced apart from each other, is arranged on an outer side of the medical instrument to be tracked, in particular on a distal end portion of the instrument, in particular integrated into the instrument, and the control unit is adapted to decode the optical pattern or to compare it with a reference stored in a storage unit and to determine a position of an instrument tip relative to the optical pattern or a geometry of the instrument on the basis of the detected optical pattern.

20. The surgical navigation system according to claim 15, wherein a geometric shape of the at least one medical instrument, in particular of several medical instruments, is stored in a storage unit, in particular by an initial three-dimensional detection by the imaging head and/or by the tracking system, and the control unit determines the position, in particular pose, of the instrument tip on the basis of a partial portion of the medical instrument detected by the imaging head and the stored geometric form.

21. The surgical navigation system according to claim 15, wherein the tracking system comprises an infrared-based camera system and/or electromagnetic-based system and/or an IMU-based tracking system.

22. The surgical navigation system according to claim 15, wherein the navigation system comprises a n image analysis device, which is adapted to perform a spatial three-dimensional detection of a pose of an instrument from at least two image perspectives, in particular a stereo image via machine vision.

23. A mobile medical navigation tower, comprising:
a surgical navigation system according to claim 15; and
a mobile cart with wheels for mobile placement of the navigation tower.

24. A navigation method for tracking of at least one medical instrument, in particular in a surgical navigation system according to claim 15, consisting of the following steps of:
registering a partial portion of a patient, in particular of the patient, with respect to 3D imaging data of the patient;
detecting and tracking the medical instrument to be tracked by an imaging head of a medical imaging device;
detecting and tracking the imaging head by a tracking system;
determining a position and/or orientation of the medical instrument, in particular an instrument tip, by linking the tracking of the imaging head and the tracking of the medical instrument;
preferably transferring the determined position and/or orientation of the medical instrument to the 3D imaging data; and
outputting a correlation representation with the 3D imaging data and with the position and/or orientation of the medical instrument by a display device.

25. The navigation method according to claim 24, further comprising the following steps of:
creating a stereo image by the medical imaging device;
creating, based on the stereo image, a depth map with depth information by triangulation and/or by reconstruction of a disparity overlap; and
determining the position and/or orientation of the instrument based on the stereo image and the depth map.

26. The navigation method according to claim 24, further comprising the following steps of:
detecting a pre-determined optical pattern, in particular a QR code and/or at least two rings spaced apart from each other as information carriers for a distance from the optical pattern to an instrument tip or for a geometry of the instrument; and
determining the position, in particular pose, of the instrument tip relative to the optical pattern based on the optical pattern.

27. The navigation method according to claim 26, wherein in the case that the pre-determined optical pattern is a QR code and a distance from the QR code to the instrument tip is encoded in the QR code, the navigation method comprises the steps of:
decoding the QR code;
reading the distance to the instrument tip; and
determining the position of the instrument tip relative to the imaging head and via the tracked imaging head relative to the 3D imaging data.

28. A computer-readable storage medium comprising instructions which, when executed by a computer, cause the computer to perform the method steps of the navigation method according to claim 24.
